(19) United States
(12) Patent Application Publication
(10) Pub. No.: US 2006/0148907 A1
(43) Pub. Date: Jul. 6, 2006

(54) Topical antinflammatory preparations of y-terpinene

(76) Inventors: David M. Nicholson, Sydney (AU); Jim Gobert, Sydney (AU)

Correspondence Address: STITES & HARBISON PLLC, 1199 NORTH FAIRFAX STREET, SUITE 900, ALEXANDRIA, VA, 22314, US

(21) Appl. No.: 10/522,198

(22) PCT Filed: Jul. 24, 2002
(86) PCT No.: PCT/AU02/00982

§ 371 Date: Aug. 14, 2005

(51) Int. Cl. A61K 31/045; A61K 31/015

(52) U.S. Cl. 514/729; 514/763

(57) ABSTRACT

An anti-inflammatory agent comprising from 0.01-1.0% γ-terpinene in a pharmaceutically acceptable vehicle.

### FIELD OF THE INVENTION

THIS INVENTION relates to topical ant-inflammatory or anti-irritant preparations for use in treatment of inflammatory disorders.

### BACKGROUND OF THE INVENTION

Reference may be made to conventional topical anti-inflammatory agents which may comprise non steroidal anti-inflammatory agents or NSAIDS which may include indomethacin, ketoprofen, piroxicam, diclofenac and benzydamine which like all NSAIDS have certain restrictions on use and, thus, should not be used with patients who are pregnant women, lactating women or patients already taking aspirin or warfarin, patents with peptic ulcers and, in some cases, on broken skin, wounds, infected skin, lesions or sores. This is pointed out in the 1997 MIMS Annual, which is published by MIMS Australia, a division of Medi Media Australia Pty Ltd. In particular reference to indomethacin, this compound is an indole derivative and is a potent inhibitor of prostaglandin synthesis, should be avoided in patients with nasal polyps and angioedema, wherein asthma may be precipitated. Indomethacin should also not be used as a suppository in patients with a recent history of proctitis and recent rectal bleeding. When indomethacin is applied topically, it can have side effects which include pruritis as described in U.S. Pat. No. 6,174,891 which may also apply to use of NSAIDS generally rashes, skin dryness and burning of skin.

Other conventional topical anti-inflammatory products include guaiazulene, which, although found in nature, is commercially produced synthetically. This product is very unstable and may discolour on standing and in formulations where guaiazulene forms an intense blue colour to the finished product. Reference also may be made to (−)-alpha-bisabolol, which is a natural product obtained from Vanillosmopsis erythropappa Schultz bip which is expensive and can be an irritant at a level of 1% or higher. There is also a synthetic version which has the same drawbacks and a lower activity.

Reference may also be made to hydrocortisone which is a topical corticosteroid useful for reducing inflammation and itch. This product should not be used on broken skin or for extended periods of time, since it can thin the skin and delay wound healing. Also prolonged use can lead to tolerance which makes the product have reduced effectiveness. Hydrocortisone in concentrations of 0.5% or less offer little benefit in therapeutic efficiency.

Reference also may be made to Hart et al, Inflammation Research 49 (2000) 619-626, which refers to the use of tea tree oil and constituents of tea tree oil as an anti-inflammatory agent In this reference it was established that the only specific report on tea tree oil being an anti-inflammatory agent was an in vitro study published as an abstract, Pippin et al, J. Dent Res. 1994 73 259, wherein it was found that the addition of tea tree oil to lipopolysaccharide primed neutrophils inhibited superoxide release by approximately 85%. The concentration of tea tree oil used was 0.05% v/v.

It was also established in Pongprayoon et al, Phytomedicine 1996-1997 3 319-322, that α-terpinene has significant anti-inflammatory activity in that it inhibited edema formation in the model of carrageenan induced hind paw edema in rats. This activity occurred at the highest dose tested which was 6 mg/paw. However, this effect was relatively weak at lesser dosages. It was also established in this reference that γ-terpinene did not exhibit any significant anti-inflammatory activity. This reference also established that terpinen-4-ol had significant anti-inflammatory activity at a range of dosages of from 0.03 mg/paw to 6.0 mg/paw. This observation was also confirmed in the Hart et al reference supra which also found that the water soluble compounds of tea tree oil, which includes terpinen-4-ol as a major component, at concentrations equivalent to 0.125% v/v significantly suppressed LPS induced production of TNFα, IL-β, 1L-10 and PGE and thus had significant anti-inflammatory activity. These water soluble components also included a-terpineol and 1,8 cineole as components in minor proportions.

However, it is noted in Tisserand and Balacs (1995) in a reference entitled “Essential Oil Safety—A Guide to Health Care Professionals” that terpinen-4-ol has an acute oral LD₅₀ of 1.3 g/kg and is described as mildly irritating and non-sensitising, a-terpinene was also found to have an acute LD₅₀ of 1.68 g/kg in contrast to the γ-isomer which was described as non-toxic, non-irritant and non-sensitising.

### SUMMARY OF THE INVENTION

It is therefore an object of the invention to provide an anti-inflammatory agent which is relatively non-toxic and effective in use.

Surprisingly, it has now been found that γ-terpinene is an effective human anti-inflammatory agent which contrasts with the observations made in prior art as discussed above and in the Pongprayoon et al reference supra which found γ-terpinene to be ineffective as an anti-inflammatory agent. The explanation may be that the rat paw model used by Pongprayoon may not be a completely effective predictor of human topical anti-inflammatories. The advantages of the use of a topical terpene anti-inflammatory agent, such as γ-terpinene, in contrast to conventional topical anti-inflammatory agents as described above include (i) the terpene is a natural product, (ii) may be produced from renewable natural resources, (iii) can be produced commercially at low cost, and (iv) is generally considered to be non toxic or having few side effects when compared to NSAIDS. In this regard, it will be appreciated that the preferred source of cosmetic raw materials for consumers are natural products and from sustainable agriculture. However, it will be appreciated that, while it is preferred that the terpenes used in the composition of the invention may be obtained as terpene fractions from tea tree oil or other essential oils, individual terpenes such as γ-terpinene or α-terpinene may be made synthetically.

Thus, the invention provides an anti-inflammatory agent which comprises from 0.01-1.0% γ-terpinene in a pharmaceutically acceptable vehicle. More preferably there is 0.05-0.5% γ-terpinene and most preferably there is 0.10-0.35% of γ-terpinene. The optimum level is 0.25%.

The anti-inflammatory agent may also include from 0.01-1.0% K-terpinene. More preferably there is 0.05-5.0% tt-terpinene and most 5 preferably there is 0.10-0.35% a-terpinene. The optimum level of α-terpinene is 0.25%. If desired, there also may be included 0.25-5.0% terpinen-4-ol and more preferably 0.75-1.25% terpinen-4-ol. The optimum level of terpinen-4-ol is 1.0%.

The most preferred formulation of the anti-inflammatory agent of the invention is a blend of 0.05-0.2% γ-terpinene and 0.05-0.2% -terpinene and such a blend may be obtained in an economical and commercially available manner by distillation of tea tree oil or other essential oils to obtain a fraction, extract or blend containing these concentrations of each of γ-terpinene and α-terpinene.

Topical products which may include the composition of the invention occur in a variety of forms, including solids, liquids, suspensions, semisolids (such as creams, gels, pastes or “sticks”), powders or finely dispersed liquids such as sprays or mists. Examples of topical products commonly classified as “cosmetics” include skin care products such as creams, lotions, moisturizers and “treatment cosmetics” such as exfolients and/or skin cell renewal agents; fragrances such as perfumes and colognes, and deodorants; shaving-related products such as creams, “bracers” and aftershaves; depilatories and other hair removal products; skin cleansers, toners and astringents; pre-moistened wipes and washcloths; tanning lotions and sunscreens; bath products such as oils; eye care products such as eye lotions and makeup removers; foot care products such as powders and sprays; skin colorant and make-up products such as foundations, blushes, rouges, eye shadows and liners, lip colors and mascaras; lip balms and sticks; hair care and treatment products such as shampoos, conditioners, colorants, dyes, bleaches, straighteners, and permanent wave products; baby products such as baby lotions, oils, shampoos, powders and wet wipe; feminine hygiene products such as deodorants and douches; skin or facial peels applied by dermatologists or cosmeticians; and others. Examples of topical products commonly classified as “topical drugs” are many and varied, and include over-the-counter and/or prescription products such as antiperspirants, insect repellents, sunscreens and sunburn treatments, anti-acne agents, antibiotics, therapeutic retinoids, anti-dandruff agents, external analgesics such as capsaicin products, topical contraceptives, topical drug delivery systems, suppositories and enemas, haemorrhoid treatments, vaginal treatments, lozenges, and many other products with therapeutic or other effects. Other topical products include hand, facial and body soaps and detergents and other forms of skin cleansers, as well as household detergents and many other household products such as solvents, propellants, polishes, lubricants, adhesives, waxes and others which are either applied topically or are topically exposed to the body during normal use.

Suitable topical vehicles for use with the compositions of the invention are well known in the cosmetic and pharmaceutical arts, and includes such vehicles (or vehicle components) as water; organic solvents such as alcohols (particularly lower alcohols readily capable of evaporating from the skin such as ethanol), glycols (such as glycerin), aliphatic alcohols (such as lanolin); mixtures of water and organic solvents (such as water and alcohol), and mixtures of organic solvents such as alcohol and glycerin (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both nonvolatile and volatile) such as cyclomethicone, demethicone and dimethicone copolyol (Dow Corning); hydrocarbon-based materials such as petrolatum and squalane; anionic, cationic and amphoteric surfactants and soaps; sustained-release vehicles such as microsponges and polymer matrices; stabilizing and suspending agents; emulsifying agents; and other vehicles and vehicle components that are suitable for administration to the skin, as well as mixtures of topical vehicle components as identified above or otherwise known to the art. The vehicle may further include components adapted to improve the stability or effectiveness of the applied formulation, such as preservatives, antioxidants, skin penetration enhancers, sustained release materials, and the like. Examples of such vehicles and vehicle components are well known in the art and are described in such reference works as Martindale—The extra Pharmacopoeia (Pharmaceutical Press, London 1993) and Martin (ed.), Remington's Pharmaceutical Sciences. The choice of a suitable vehicle will depend on the particular physical form and mode of delivery that the formulabon is to achieve. Examples of suitable forms include liquids (as well as suspensions, emulsions and the like); solids and semisolids such as gels, foams, pastes, creams, ointments, “sticks” (as in lipsticks or underarm deodorant sticks), powders and the like; formulations containing liposomes or other delivery vesicles; rectal or vaginal suppositories, creams, foams, gels or ointments; and other forms. Typical modes of delivery include application using the fingers; application using a physical applicator such as a cloth, issue, swab, stick or brush (as achieved for example by soaking the applicator with the formulation just prior to application, or by applying or adhering a prepared applicator already containing the formulation—such as a treated or pre-moistened bandage, wipe, washcloth or stick—to the skin); spraying (including mist, aerosol or foam spraying); dropper application (as for example with ear drops); sprinkling (as with a suitable powder form of the formulation); and soaking.

Methodologies and materials for preparing formulations in a variety of forms are also described in Anthony L. L. Hunting (ed.), “A Formulary of Cosmetic Preparations (Vol. 2)—Creams, Lotions and Milks,” Micelle Press (England, NI.J. 1993). See, for example, Chapter 7, pp. 5-14 (oils and gels); Chapters, pp. 15-98 (bases and emulsions); Chapter 9, pp. 101-120 (“all-purpose products”); Chapter 10, pp. 121-184 (deansing masks, creams, lotions); Chapter 11, pp. 185-208 (foundation, vanishing and day creams); Chapter 12, pp. 209-254 (emollients); Chapter 13, pp. 297-324 (facial treatment products); Chapter 14, pp. 325-380 (hand products); Chapter 15, pp. 381-460 (body and skin creams and lotions); and Chapter 16, pp. 461-484 baby products; the contents of which are incorporated herein by reference.

In another embodiment of the invention, the use of the invention can be formulated in a form for topical oral administration to treat pain or irritation in the mouth or throat such as that due to sore throats, canker sores, gum irritation or inflammation or the like, including such irritation as may be exacerbated by spicy or acidic foods. Methods for preparing oral formulations suitable for use In the present invention are well known in the art,

Antioxidant Activity

As terpenes exhibit antioxidant activity they may oxidise as reported in Ruberto etal, Food Chemistry 69 (2000) 167-174, Therefore, it is useful to include with the composition of the invention antioxidants, inclusive of alpha-tocopherol or beta hydroxy toluene, or use encapsulation techniques which include liposomes, cyclodextrins, maltodextrins or melamine to protect and stabilise the terpenes combined in the composition of the invention. Other materials that may be used to encapsulate the terpenes are zeolites and polyamides. Such encapsulation techniques may be utilised to provide a controlled release or body responsive effect where the encapsulation dissolves in perspiration or physically breaks to release the active ingredient. Such encapsulation techniques may also be utilised to protect the active ingredient from a chemically damaging medium such as talc (where the high surface are may render the active ingredient susceptible to oxidation) or antiperspirants (where the normal antiperspirant actives such as aluminium chlorhydrate may decompose the active ingredient).

In support of the foregoing, reference may be made to U.S. Pat. No. 6,187,351 which is herein incorporated by reference to provide background techniques concerning encapsulation of the terpene actives to prevent oxidation. The use of liposomes is discussed in U.S. Pat. Nos. 6,083,529 and 6,132,766 which are also incorporated herein by reference. Reference also may be made to cyclodextrins in U.S. Pat. Nos. 6,025,510, 5,985,296 and 5,879,692 which are incorporated herein by reference. Controlled release formulations are described in U.S. Pat. No. 6,129,931 which is also incorporated herein by reference.

Numerous processes are known in the art for producing microencapsulated materials. Nearly all the known process produce microcapsules of materials contained in a water-immiscible or insoluble material and are produced by what is termed oil-in-water microencapsulation processes. These in general involve the production of a dispersion of “oil” or organic, substantially water-immiscible liquid droplets (discontinuous phase) in an aqueous medium (continuous phase). The oil droplets contain one or more monomers or prepolymers and microcapsules are formed by subjecting the emulsion to conditions such as temperature and/or pH and/or agitation to cause polymerization of the monomers or prepolymers present in the oil phase to produce microcapsules having a polymeric shell enclosing the water-immiscible droplet phase. Such processes are described, for example, in U.S. Pat. Nos. 4,285,720 and 4,956,129 which are incorporated herein by reference. The former involves production of microcapsules of a polyurea material and the latter of an etherified urea-formaldehyde polymer.

Reference also may be made to the inclusion of skin whitening products within the compositions of the invention to reduce permanent pigment darkening as well as increasing SPF by reduction of erythemal response. Reference may be made to U.S. Pat. No. 6,139,856 in this regard which is also incorporated herein by reference.

It will also be appreciated that the compositions of the invention apply to veterinary products as well as for application to humans. The formulation may be in any conventional form suitable for use as a cosmetic, pharmaceutical product or personal care product. The composition may also include additives and excipients conventionally found in topical formulations, such as emulsifiers, surfactants inclusive of ionic, non-ionic and amphoteric surfactants, thickening agents, emollients, stabilizers and humectants.

The composition of the invention may be used as a clear aqueous solution which may comprise, in addition to the above active components, 0.1-20% surfactant, emulsifier or solubilizing agent. More preferably a non-ionic surfactant such as PEG-35 Castor-Oil may be utilized. Other suitable surfactants are also described hereinafter.

In addition to the aforementioned surfactant, the composition of the invention may include 1-10% of an emollient which moisturizes the skin or, more preferably, 1-5% of the emollient A suitable emollient is PEG 7 glyceryl cocoate. There also may be included 1-10% and, more preferably, 1-5% of a humectant, such as glycerol or propylene glycol and 1-5% of a thickener or viscosity increasing agent, such as a gum, in the form of a guar gum, gum tragacanth, xanthan gum, galactomannan gum or a polyacrylic acid. There also may be included 1-15% of a detergent, such as sodium lauryl ether sulphate and/or ammonium lauryl sulphate. There also may be provided from 1-5% of a cleaning agent, such as coconut diethanolamide. When used as a cream, the composition may include waxes, such as 1-5% of cetyl alcohol or stearyl alcohols. There also may be included essential oils, herbs, vitamins, such as Vitamin E or Vitamin A, hydrolysed collagen, amino acids, panthenol and other nutritional factors as may be required. Examples of compositions of the invention when used as a clear aqueous solution, clear liquid soap, moisturizing cream with collagen and herbal extracts, a pearlescent shampoo and a hair conditioner are set out herein below.

(A) Clear Aqueous Solution

0.125%—γterpinene, 0.125% α-terpinene, 1.6-3.2%

Polysorbate or other suitable non-ionic surfactant, 0.1% disodium edetate or other suitable chelating agent and 0-5.0% butylene glycol with the balance being purified water.

(B) Clear Liquid Soap

0.125% γ-terpinene, 0.125% α-terpinene, 10.0% sodium lauryl ether sulphate 70.0% (Empicol ESB 70), 5.0% ammonium lauryl sulphate 30.0% (Empicol ALS 30), 0.05% disodium edetate, 1.5% cocobetaine (Empigen BB), 1.9% coconut diethanolamide (Empilan FD), 0.20% sodium chloride, citric acid in 10.0% solution to provide a pH of 6.7 and the balance purified water.

(C) Moisturizing Cream with Collagen

0.125% γ-terpinene, 0.125% α-terpinene, 5.0% octyl palmitate, 1.0% olive oil, 4.0% jojoba oil, 1.0% macadamia nut oil, 4.0% cetearyl glucoside (Montanol 68), 0.3% natural Vitamin E (Covitol F1300), 0.075% Vitamin A palmitate, 2.0% butylene glycol, 5.0% propylene glycol, 1.0% hydrolysed collagen, 10.0% aloevera gel, 0.1% disodium edetate, perfume 0.35% and the balance purified water having a pH of 5.1.

(D) Pearlescent Shampoo

0.125% γ-terpinene, 0.125% α-terpinene, 10% sodium lauryl ether sulphate, 0.05% disodium edetate, 3.0% cocobetaine, 3.0% coconut diethanolamide (Empilan FD), 0.10% Polyquatemium (Polymer JR-400), 3.0% pearling agent (Euperian PK771), 0.20% sodium chloride, citric acid (10.0%) to make up a pH of 6.5 with the balance being purified water. (E) Hair conditioner 0.125% Y-terpinene, 0.125% a-terpinene, 3.0% cetostearyl alcohol, 0.15% Dimethicone Vitamin E Natural (Covitol F1300), 0.05% PEG-7 glyceryl cocoate (Cetiol HE), 1.0% wheat protein amino acids (Hydrotriticum WAA), 0.10% citric acid (10.0%), 0.10% panthenol and 1.0% cetyltri methyl ammonium chloride 50.0% (Dehyquart A) with the balance being purified water.

The surfactant referred to above may be an anionic surfactant, such as a carboxylate, sulfonate, sulfated alcohol or sulfated alcohol ethoxlate. Cationic and amphoteric surfactants may also be used but the preferred surfactant is a non-ionic surfactant, such as polyoxyethylene surfactant or carboxylic acid esters, such as glycerol esters, polyoxyethylene 5 esters, anhydrosorbitol esters, natural fats, oils and waxes and ethoxylated and glycol esters of fatty acids.

### EXPERIMENTAL

Human Study Overview

Purpose: The purpose of the study was to evaluate the anti-10 inflammatory effectiveness of several materials by determining their ability to reduce the skin erythemal response induced by solar simulated UV on the dorsal skin of human subjects.

Venue of Study: Australian Photobiology Testing Facility, Ross Street Building, Sydney University.

UV Source: Solar Light Co, Phil, USA 1 SOW, 16S single port Solar simulator—spectrum conforming to limits set in Australian standard.

Methodology: Testing conducted on 31 human subjects.

Day 1: Minimum erythemal dose (MED) assessment—each subject receives a series of UV exposures at one-second increments on 20 unprotected skin to determine MED 16-24 hours later.

Day 2: MED determined by reading irradiated skin. Additional irradiation carried out at three levels:
irradiated to below one MED;
irradiated at one MED;
irradiated at above one MED.

Test product applied at 4 mg/sq cm onto skin immediately after irradiation then again 4-5 hours later.

Day 3: 16-24 hours later erythema observed, measured and photographed. Results subsequently were compiled, analysed and confirmed using REML analysis of variance. The inflammation Reduction (IR) was calculated as the reduction in erythema compared with base.

Notes: Base used was same for all products—BP 1988 Cetomacrogol Cream.

Negative Controls—UV irradiation only; base vehicle.

Positive Control—1% Indomethacin BP88 (a non steroidal anti-inflammatory).

### RESULTS

Table 1 summarises the rank order of anti-inflammatory effectiveness in a typically “unbalanced” study where there are different numbers of subjects per product/group. Of the total number of 310 measurements from 31 subjects and 17 products, 200 remained after the application of inclusion and exclusion criteria. Subjects form whom responses to the BASE were both equal to or more than the MED, and less than the MED but greater than Indomethacin, were included, and those for whom the responses to the BASE were less than the MED and equal to or less than Idomethacin, were excluded. The 110 measurements on 1 subjects excluded were accorded the status of “non-responders”. The mean number of “responders” remaining per productitest group was 10 with group sizes ranging from 4 to 20. This variability may merely reflect that of a normal diversity of the human population. The analysis used was a subject product two-way analysis of variance with subjects regarded as a random effect. The overall results were confirmed using a REML (Restricted Maximum Likelihood) analysis of variance. Given that the visual scores do not follow a normal distribution, the subsequent p-values should be treated with some caution: actual relative differences of products may well be important even though the current p-values based on the current samples may be larger than 0.05.

The Positive and Negative Controls: UV alone (MED), compared with Base, and Indomethacin were significantly different or not different in a way entirely consistent with expectations. Indomethacin was the most effective anti-inflammatory, the MED was the most erythemally effective, ad the Base ranked with the MED and without any statistically significant difference.

0.25% γ-terpinene, 0.50% alpha-bisabolol, 0.25% Blend, and 1.0% tea tree oil were statistically different (p-<0.05) from the Base and UV 5 alone (MED). These products permitted between 40% and 98% more erythema than the Indomethacin (UV alone permitted 224% more erythema); between 52 and 33% less erythema than the Base, and between 57 and 39% less erythema than with UV alone. 0.25% γ-terpinene, 0.50% alpha-bisabolol and 1.0% tea tree oil might be considered similar in effectiveness 10 to Indomethacin (no significant difference at the 5% level). 1.0% terpinen-4-ol and 0.25% α-terpinene while limited for evaluation by their small sample sizes must be accorded a good performance status. 1.0% γ-terpinene was not significantly different from the Base and permitted 98% more erythema than Indomethacin while reducing UV erythema (MED) by 39%. This product was significantly different from the MED but statistically similar to the effectiveness of Indomethacin. It is because logic would then insist that the Base and Indomethacin are therefore similar, which is inconsistent with the direct comparison of Base and Indomethacin, subject variability may account for this anomaly.

### DISCUSSION

The results indicate that the “Blend” at 0.25% (i.e. γ-terpinene 0.125% and α-terpinene fraction 0.125%) is a high effective anti-inflammatory compared to Indomethacin 1% and a-bisabolol 0.5%. The same conclusion applies to 0,25% γ-terpinene.

The “Blend” was a blend of natural terpenes including γ-terpinene and α-terpinene obtained from fractional distillation of tea tree oil in approximately the levels contained in tea tree oil (tea tree oil contains approximately 30% of the blend—yet the Blend at 0.25% had an Inflammation Reduction 40% whilst tea tree oil at 1% had an Inflammation Index of 33%). Consequently, the “Blend” had an unexpectedly strong anti-inflammatory effect. From this research conducted, the anti-inflammatory effect is expected to be the same for α-terpinene and γ-terpinene and the “Blend” whether of natural or synthetic origin and in varying blended percentages. 5 There is no indication of synergy between α-terpinene and γ-terpinene. This study indicates that at higher levels, the Inflammation Index for the blend (and associated ingredients) is lower. This is not a totally unexpected result as other anti-inflammatory ingredients are known to display similar properties (e.g. at 1% α-bisabolol is considered an inflammation promoter by some researchers).

### APPLICATION TO CONSUMER PRODUCTS

The results of testing on human subjects demonstrates that the materials “Blend” and α-terpinene and γ-terpinene are effective anti-inflammatories when applied to human skin which has been, subject to inflammation. Application to several product types for use on inflamed skin logically immediately follows including after sun products (source of inflammation being UV radiation), after shave products (source of inflammation being razor, drag and/or soap), babycare i.e. “nappy rash” products (source of inflammation being ammonia), burn care and insect bite care.

It is expected that these materials will reduce the inflammation response when incorporated into consumer products which have components which induce inflammation on the skin (i.e. incorporated so that the anti-inflammatory agent is applied to the skin in the product incorporating the ingredient which promotes skin inflammation). Products of this type include anti-perspirants (inflammation promoter being aluminium chlorhydrate), permanent wave lotions and depilatories (inflammation promoter being ammonium thioglycollate and related compounds) and skincare products containing alpha-hydroxy acids and beta hydroxy acids, and insect repellants.

It is expected that these materials, although not UVB absorbing chemicals, when incorporated into sunscreens will increase the in vivo SPF by inhibiting the erythemal response (as SPF is a numerical ratio based on erythemal response in human subjects).

It also follows that in some countries (e.g. Japan), the measurement of UVA protection is determined by assessing the permanent pigment darkening which is UVA dependent and proceeds, inter alia, via a free radical reaction mechanism. As these terpene products have antioxidant/free scavenging radical effects, they may consequently improve the measured UVA protection although not having any UVA absorbance in themselves. It is expected that the anti-inflammatory properties of these materials would be of benefit in a range of pharmaceutical products including first aid treatments (for bums etc), and haemorrhoid control.

TABLE 1

| Sample | Reduction in Erythema vs Base |
|---|---|
| 1% Indomethacin | 66% |
| 0.25% α-terpinene | 58% |
| 0.25% γ-terpinene | 52% |
| 0.5% Terpinen-4-ol | 45% |
| 0.5% (−) alpha-Bisabolol | 44% |
| 0.25% “Blend” | 40% |
| 1.0% MC PG Tea Tree Oil | 33% |
| 1.0% γ-terpinene | 32% |
| 0.5% γ-terpinene | 29% |
| 0.5% “Blend” | 26% |
| 1.0% “Blend” | 15% |
| 0.25% (−) alpha-Bisabolol | 10% |
| 0.25% Terpinen-4-ol | 7% |
| 0.5% MC PG Tea Tree Oil | 6% |

1. An anti-inflammatory agent comprising from 0.01-1.0% γ-terpinene in a pharmaceutically acceptable vehicle.

2. An anti-inflammatory agent as claimed in claim 1 comprising 0.05-0.5% γ-terpinene.

3. An anti-inflammatory agent as claimed in claim 1 comprising 0.05-0.2% γ-terpinene.

4. An anti-inflammatory agent as claimed in claim 1 comprising 0.10-0.35% γ-terpinene.

5. An anti-inflammatory agent as claimed in claim 1 comprising 0.25% γ-terpinene.

6. An anti-inflammatory agent as claim 1 further comprising 0.25-5.0% terpinen-4-ol.

7. An anti-inflammatory agent as claimed in claim 6 further comprising 0.75-1.25% terpinen-4-ol.

8. An anti-inflammatory agent as claimed in claim 7 further comprising 1.0% terpinen-4-ol.

9. An anti-inflammatory agent as claimed in claim 1 further comprising 0.05-0.2% α-terpinene.

10. An anti-inflammatory agent as claimed in claim 1 comprising a blend of 0.05-0.2% γ-terpinene and 0.05-0.2% α-terpinene.

11. An anti-inflammatory agent as claimed in claim 1, wherein the pharmaceutically acceptable vehicle is water.

12. An anti-inflammatory agent as claimed in claim 1, wherein the pharmaceutically acceptable vehicle is an organic solvent.

13. An anti-inflammatory agent.as claimed in claim 1, wherein the pharmaceutically acceptable vehicle is a mixture of organic solvent and water.

14. An anti-inflammatory agent as claimed in claim 1, wherein the pharmaceutically acceptable vehicle is a lipid-based material.

15. An anti-inflammatory agent as claimed in claim 1, wherein the pharmaceutically acceptable vehicle is a protein-based material.

16. An anti-inflammatory agent as claimed in claim 1, wherein the pharmaceutically acceptable vehicle is a silicone-based material.

17. An anti-inflammatory agent as claimed in claim 1, wherein the pharmaceutically acceptable vehicle is a hydrocarbon-based material.

18. An anti-inflammatory agent as claimed in claim 1, wherein the pharmaceutically acceptable vehicle is selected from an anionic, cationic and amphoteric surfactant or a soap.

19. An anti-inflammatory agent as claimed in claim 1, wherein the pharmaceutically acceptable vehicle is a sustained release vehicle.

20. An anti-inflammatory agent as claimed in claim 1, wherein the pharmaceutically acceptable vehicle is selected from a stabilising or suspending agent or emulsifying agent.

21. An anti-inflammatory agent as claimed in claim 1 in the form of an agent suitable for topical oral administration.

22. An anti-inflammatory agent as claimed in claim 1 further including an anti-oxidant selected from α-tocopherol or β-hydroxy toluene.

23. An anti-inflammatory agent as claimed in claim 1 including an encapsulating agent selected from liposomes, cyclodextrins, maltodextrins, melamine, zeolites and polyamides.

24. An anti-inflammatory agent as claimed in claim 1 further comprising 0.1-20% surfactant.

25. An anti-inflammatory agent as claimed in claim 1 further comprising 1-10% of an emollient.

26. An anti-inflammatory agent as claimed in claim 1 further comprising 1-10% of a humectant.

27. An anti-inflammatory agent as claimed in claim 1 further comprising 1-5% of a thickener or viscosity increasing-agent.

28. An anti-inflammatory agent as claimed in claim 1 further comprising 1-15% of a detergent.

29. An anti-inflammatory agent as claimed in claim 1 further comprising 1-5% of a cleaning agent.

30. An anti-inflammatory agent as claimed in claim 1 further comprising 1-5% of a wax.
